(19) United States
(12) Patent Application Publication
(10) Pub. No.: US 2012/0290099 A1
(43) Pub. Date: Nov. 15, 2012

(54) MINIMALLY INVASIVE SURGICAL TOOLS FOR HIP PROSTHESIS

(75) Inventors: Luke Andrew Gibson, Southaven, MS (US); William L. Waltersdorff, Hernando, MS (US)

(73) Assignee: SMITH & NEPHEW, INC., Memphis, TN (US)

(21) Appl. No.: 13/002,969

(22) PCT Filed: Jul. 8, 2009
(86) PCT No.: PCT/US09/49981

§ 371 Date: Jan. 6, 2011

(30) Foreign Application Priority Data
Jul. 8, 2008 (US) 61079011

(51) Int. Cl. A61F 2/38; A61B 17/56

(52) U.S. Cl. 623/20.11; 606/86 R

(57) ABSTRACT

Instrumentation for a modular prosthesis having a stem and a neck comprises a handle and an adaptor. The handle extends along an axis. The handle has a distal end and a proximal end. The proximal end has a blunt surface configured to be struck. The adaptor has a male end configured to mate inside the bore of the stem. The adaptor further has a surface configured to mate with the distal end of the handle. The adaptor is configured to have a medial portion and a lateral portion. The medial portion has a relief such that the adaptor is configured to disengage the stem when the adaptor is rotated relative the stem.

### CROSS-REFERENCE TO RELATED APPLICATIONS

This application claims the benefit of U.S. Provisional Application No. 61/079,011, filed Jul. 8, 2008. The disclosure of each application is incorporated by reference in its entirety.

### BACKGROUND OF THE INVENTION

1. Field of the Invention

This invention relates generally to minimally invasive surgical tools for hip prostheses and, more particularly, to surgical tools for modular hip prostheses.

2. Related Art

Currently, there is a need to reduce surgical time and provide less-invasive means for implanting surgical devices. In particular, there is a need for improved instrumentation for hip stems which use modular-style necks. There is also a need to trial from a postless broach that uses modular-style necks. There currently exists a challenge in allowing the broach to be properly inserted and extracted under great forces without the use of a conventional “post” or “locking shank”. To date, it is believed that the only manufacturers using modular necks are Cremescoli (now Wright Medical), and Braun, which has the METHA modular short stem. It is also believed that the instrumentation associated with prior art Cremescoli hip prosthetics does not possess the novel features of the present invention described below (e.g., ability to use conventional broach handles with modular neck systems).

Plus Orthopaedics (now a part of Smith & Nephew) has provided a postless shoulder (humerus) broach. The broach comprises a seemingly frustoconically tapered hole and a proximal periphery suitable for engagement by a broach handle. The broach handle comprises a mechanism that externally engages said proximal periphery of the broach. However, said postless shoulder broach is not adapted to receive a modular-style neck, and the external engagement means may be considered generally more invasive than the purely internal engagement means provided by the present invention.

US 2007/0043445 and related case WO 05060877 discloses an assembly consisting of a movable neck for a hip prosthesis being provided with a tapered part to aid in extraction.

### SUMMARY OF THE INVENTION

It is in view of the above problems that the present invention was developed.

Some embodiments of the present invention are drawn to an instrumentation kit for an MIS hip procedure using a hip stem with a modular neck. The kit may generally comprise tools which may be any one or more of the following: a neck extractor; a stem inserter; a combination of a stem inserter and extractor; a combination of a neck extractor stem inserter, and stem extractor; a stem adaptor; a post-less broach inserter; a postless broach handle; and combinations thereof. The individual parts of the instrumentation kit are broken down in detail below.

Some embodiments of the present invention afford interchangeability of MIS surgical instruments with conventional instruments, thereby eliminating the need to have more tools in an instrument kit. The present invention also provides instrumentation for efficiently installing implant stems and broaches that are configured to receive modular necks. Therefore, the surgical procedure is less invasive, takes less time to complete, and requires less instrumentation.

Some embodiments of the present invention aim to solve the aforementioned problems by creating a postless broach and instrumentation associated therewith that will: 1) accept a trial neck very similar or exactly like the one used for the implant to be used, 2) be able to be inserted and extracted easily without the use of a conventional broach post, and 3) be strong enough and robust enough to prevent breakage. Embodiments of the present invention also aim to enable a user to trial using a broach that is maintained in the medullary canal, and then trial again (as a double/final check) when the final implant is maintained in the medullary canal. Trialing off the broach obviates the need to remove the broach for trial reduction, and further obviates the need for a separate “trial implant” in the instrumentation system. By eliminating the middle step of providing a trial implant for trial reduction, overhead and overall bulkiness of the instrumentation kit is reduced. Embodiments of the present invention also provide instrumentation for easily removing modular necks from implant stems or broaches as needed in a minimally-invasive way.

In another aspect of the invention, instrumentation for a modular prosthesis having a stem and a neck comprises a handle and an adaptor. The handle extends along an axis. The handle has a distal end and a proximal end. The proximal end has a blunt surface configured to be struck. The adaptor has a male end configured to mate inside the bore of the stem. The adaptor further has a surface configured to mate with the distal end of the handle. The adaptor is configured to have a medial portion and a lateral portion. The medial portion has a relief such that the adaptor is configured to disengage the stem when the adaptor is rotated relative the stem.

In yet other aspects of the invention, the relief may be a radius. The distal end may extend from the handle at an angle to the long axis. The handle may be configured to have a slot extending away from the long axis and the adaptor surface configured to mate with the distal end of the handle is a flange. The adaptor may further be configured to have a slit between the medial portion and the lateral portion. The slit may be configured to flex the medial and lateral portions of the adaptor such that the adaptor is wedged within the bore when the adaptor is inserted within the bore. The handle may be an extraction tool. The handle may be an insertion tool. The adaptor may be a hollow body.

In yet another embodiment of the invention, an extraction tool for extracting a modular neck from a stem of a prosthetic device comprises a proximal end, a shank and a distal end. The proximal end has a blunt surface configured to be struck. The shank extends along an axis away from the proximal end. The distal end extends from the shank at an angle to the long axis. The distal end is forked such that a pair of prongs on the fork may engage the modular neck seated in the stem. The prongs have a first distance at a point close to the long axis and a second distance greater than the first distance at a point away from the long axis relative to the first point.

According to other embodiments, the distal end may narrow as the distal end extends away from the shank. The extraction tool may wedge the neck between the prongs when the blunt surface is struck. The extraction tool may deliver a first force to the stem generally along the long axis of the handle and a second force between the extraction tool and the neck generally perpendicular to the long axis such that the neck is forced away from the stem. The extraction tool may be rotated relative to the stem to force the neck away from the stem.

Another aspect of the invention provides a method for performing a prosthetic surgery including a modular stem having a bore configured to receive a neck component. A step couples the bore of the modular stem to a male mating surface on a distal end of a first elongated tool having a blunt proximal end configured to be struck. Another step inserts the stem into a reamed bone canal. A striking step strikes the blunt proximal end of the tool to seat the stem. Another step rotates the first elongated tool relative to the stem to decouple the tool from the stem.

Yet other aspects of the invention may provide removing a neck component from the stem by striking the blunt proximal end of the tool thereby delivering a first force to the stem generally along a long axis of the tool and a second force between the tool and the neck component generally perpendicular to the long axis such that the neck is forced away from the stem. Additionally, the method may provide the step of wedging the tool against the neck component by striking the blunt proximal end. The method may further comprise the step of removing a neck component from the stem by rotating the tool relative to the stem.

Further features, aspects, and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate embodiments of the present invention and together with the description, serve to explain the principles of the invention. In the drawings:

FIG. 1 is a view of a neck extractor of an embodiment of the present invention;

FIG. 2 is a see-through view of part of the neck extractor of FIG. 1, a modular neck and a stem;

FIG. 3 is a view of a part of the neck extractor of FIG. 1;

FIG. 4 is a view of a part of the neck extractor of FIG. 1 and a stem with a modular neck in a bone;

FIG. 5 is another view a part of the neck extractor of FIG. 1 and a stem with a modular neck in a bone;

FIG. 6 is another view a part of the neck extractor of FIG. 1 and a stem with a modular neck in a bone;

FIG. 7 is another view a part of the neck extractor of FIG. 1 after pulling a neck from a stem in a bone;

FIG. 8 shows views from different orientations of a stem adaptor;

FIG. 9 is a view of an alternate embodiment of a stem adapter;

FIG. 10 is a view of an alternate embodiment of a stem adapter;

FIG. 11 is a view of an alternate embodiment of a stem adapter;

FIG. 12 shows views of a stem inserter;

FIG. 13 shows views of a stem inserter receiving a stem adaptor;

FIG. 14 is a view of a stem inserter and adaptor engaged to a stem inserter tool;

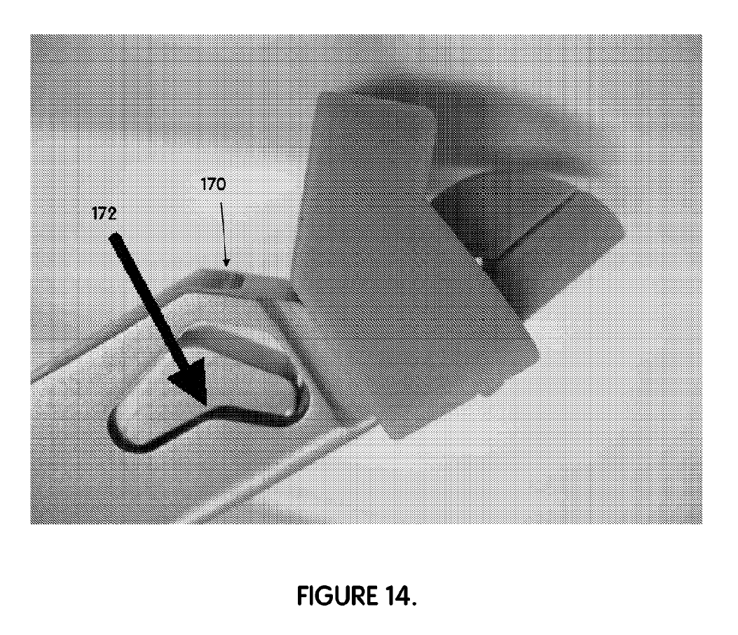

FIG. 15 is a view of a stem inserter and adaptor engaged to a stem inserter tool ready to be inserted into a stem;

FIG. 16 is a view of a stem attached to a stem inserter tool;

FIG. 17 is a view of a stem attached to a stem inserter tool, inserted in an intramedullary canal;

FIG. 18 is a view of a stem adaptor being removed from a stem;

FIG. 19 is a view of a stem properly inserted in an intramedullary canal

FIG. 20 shows views of a stem, an alternative stem adaptor, and a stem inserter;

FIG. 21 is a view of an alternate embodiment of a stem, stem inserter and stem adaptor including a fixation element;

FIG. 22 is a view of the stem inserter and stem adaptor of FIG. 21;

FIG. 23 is a view of a stem adaptor coupled to a stem extractor;

FIG. 24 is a view of the stem adaptor and neck extractor of FIG. 23 and a stem;

FIG. 25 is a see-through view of the stem adaptor and neck extractor of FIG. 24 coupled to a stem; and

FIG. 26 shows views of an alternate embodiment of a stem inserter with a positive mechanical engagement element.

### DETAILED DESCRIPTION OF THE EMBODIMENTS

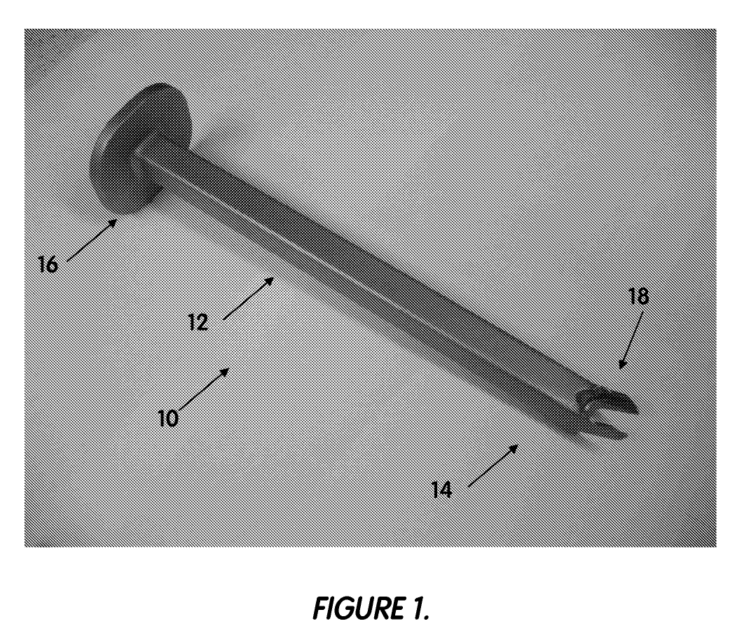

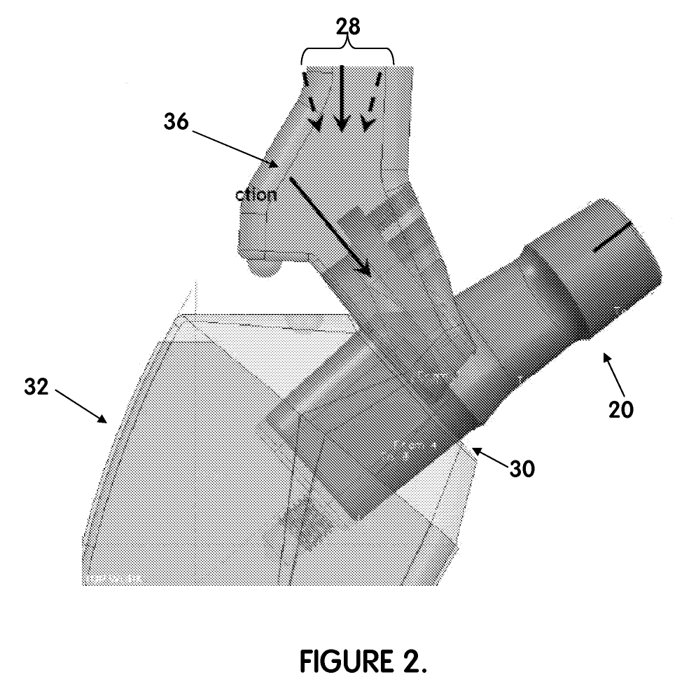

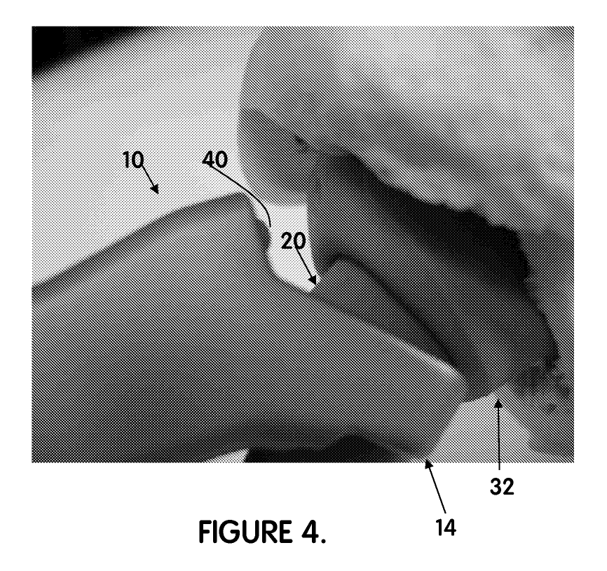

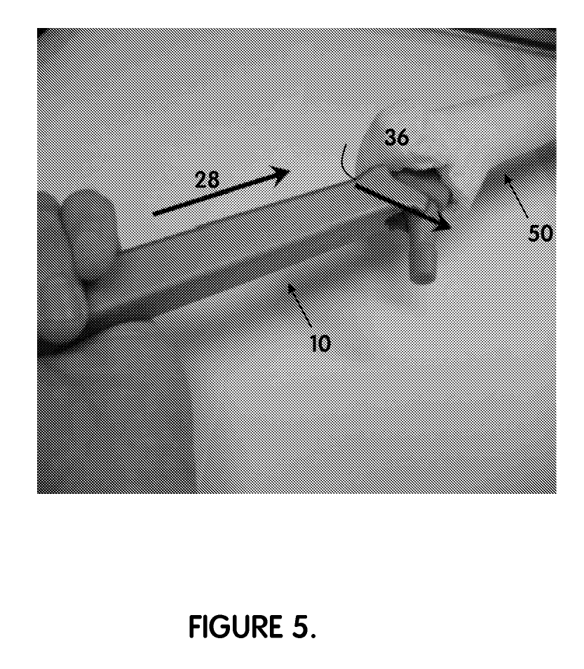

Referring to the accompanying drawings in which like reference numbers indicate like elements, FIGS. 1 through 7 illustrate a neck extractor 10. The neck extractor 10 generally comprises an elongated neck extractor shank 12 having a bi-forked distal end 14 and a blunt proximal end 16. The bi-forked distal end 16 may have projections 18 configured to extend from the shank 12 to grasp a neck implant 20 (FIG. 2).

The bi-forked distal end 14 preferably comprises a wedge shaped when viewed in a side profile. However, the bi-forked end 14 may also comprise a curved or flattened shape when viewed in a side profile. Such shape may be beneficial in inserting the forked end around the neck 20.

Preferably, the inside surface of each extending projection 18 comprises a uniform radius or chamfer. However, the radius/chamfer may vary or “grow” in a direction from a projection 18 toward the elongated shank 12 to enhance “wedging”. The inside surfaces of each finger may be closer to each other adjacent the elongated shank 12 than at the distal end of the bi-forked end 14 to additionally provide a means for wedging and captivating the modular neck 20 once removed from a stem or broach. Moreover, a lip or other protuberance or ridge may be provided at the bottom or distal portion of each of said two extending fingers to better engage the modular neck 20.

In use, the neck extractor 10 may first be lined up such that the modular neck 20 extends up and through a distal portion of the bi-forked end 14 between each projection 16. Depending on tolerances, in use, the modular neck 20 may be located anywhere along the length of the projections 18 before becoming initially “wedged” between said fingers. In the case of larger modular necks, the wedging might initially occur at a more distal portion of the bi-forked end 14. In the event of a smaller modular neck, initial wedging may occur between the fingers further inboard from a distal portion of the bi-forked end 14.

The neck extractor 10 may be impacted at its blunt end 16 at any angle generally as long as a substantial force vector component 28 of the blow is directed along the axis of the elongated shank 12. The “wedge” shape of the bi-forked end, in combination with an angled proximal face 30 of a stem body 32 (which may be an implant, trial implant, or a broach stem body), translates much of the axial forces of the blow to a more transverse “sliding” motion 36 of the bi-forked end 14 along the angled proximal face 30 of the stem body 32 at the junction between the modular neck 20 and stem body 32. The wedge-shape of the bi-forked end 14 provides a “pulling” force to a peripheral surface of the modular neck as it translates, which ultimately dislodges the modular neck from the implant stem/trial or broach.

Depending on how tight the modular neck fit is within a bore in the stem body 32, a neck extractor 10 may or may not be needed for trial reduction. Generally speaking, it may be needed in a revision procedure to remove a previously impacted modular neck since oxidation may occur over time and increase the forces required for neck removal. The neck extractor 10 of the present invention is configured to remove a modular-style neck from a femoral stem body in a minimally invasive manner.

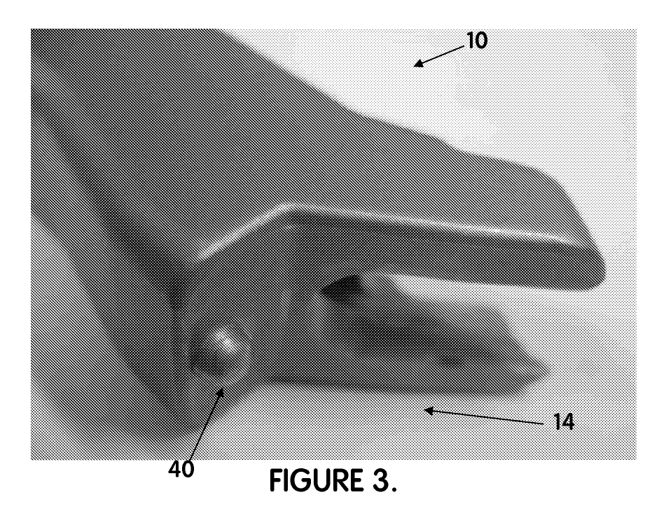

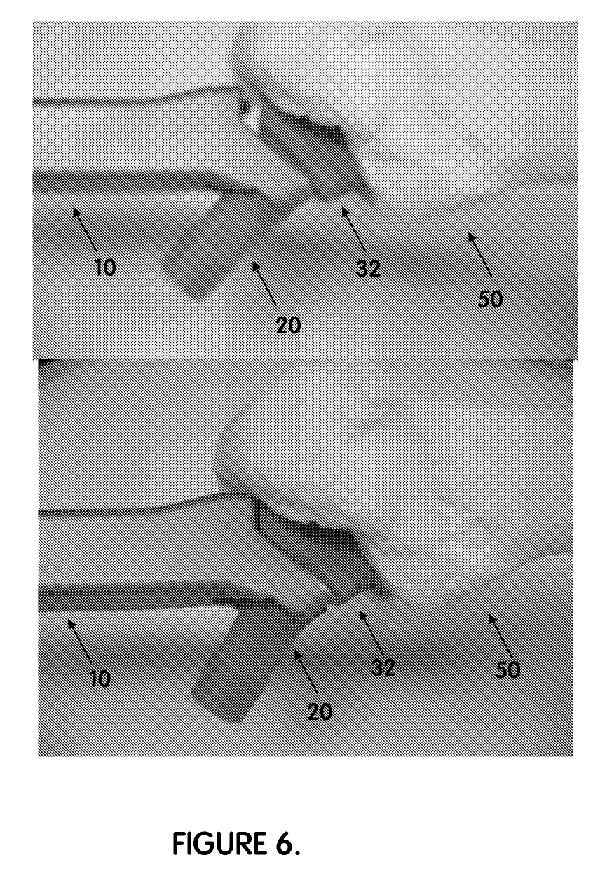

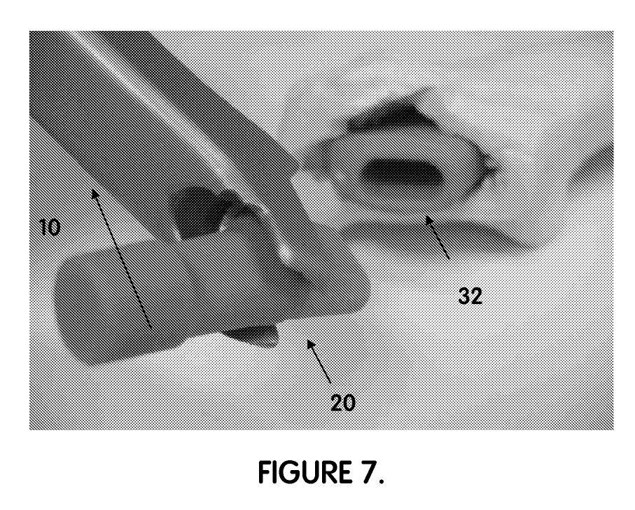

As shown in FIG. 3, the extractor 10 may also have an indent 40. The indent 40 may align the extractor 10 along the proximal face 32. Alignment, improperly shown on the top portion of FIG. 6 and properly shown on the bottom portion of FIG. 6 shown may help to pull the neck 20 from the stem 32. The neck 20, then, as shown in FIG. 7, may dislodge free from the stem 32 and be held in the projections 18 of the extractor 10.

The translation motion that pulls the neck 20 from the stem 32 creates a relative force between the stem 32 and the neck 20 by moving the extractor 10 along the surface 30. This may minimize extraction forces between the stem 32 and bone 50.

While it is preferred that the neck extractor has a wedge-shaped geometry between its fingers to help “pull” the neck out using only a small impact, the neck extractor may omit such a wedge-shape, and may instead comprise fingers having flat parallel lips. After impaction, the surgeon may “lever” the neck out to replace the wedge function.

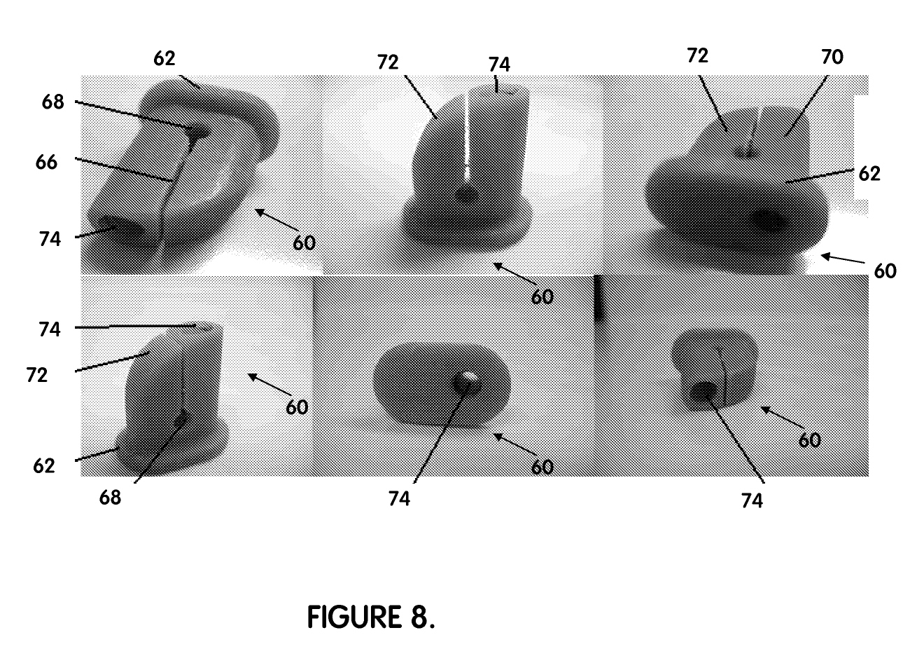

FIG. 8 shows views from different orientations of a stem adaptor 60. The stem adaptor 60 may comprise a flange portion 62 connected to a leg portion 64. The leg portion 64 may include a longitudinally-extending flexion slit 66 and a flexion hole 68. The flexion slit forms two prongs, a lateral prong 70 and a medial prong 72, one of which is preferably a chamfered prong 72 or a radiused prong 72. The non-radiused prong 70 comprises a longitudinally extending bore 74 through its entire length. The bore has a proximal end which extends through the flange, and a distal end that terminates at the extremity of the prong 70. The bore 76 allows passage of a fixation means (e.g., a screw or the like) therethrough.

In a preferred embodiment, the stem adaptor 60 is made from a plastics material which may or may not be disposable, since plastic is unlikely to scratch the implant, broach, or trial implant (if used). However, it can be formed of any suitable material such as a bio-compatible metal (e.g., stainless steel) or another hard material which is provided with a surface that resists scratching (e.g., rubber or polymer overmoulded onto steel). The leg portion 64 of the stem adaptor 60 is configured to fit snugly into a preferably oval-shaped, Morse-tapered bore in a stem of a postless broach, implant, or trial implant. Such a bore is configured to accept modular-style necks of various sizes, offsets, and version. The flexion slit 66 and flexion hole 66 provided on the stem adaptor 60 allow the prongs 70 and 72 of the leg portion 64 to squeeze together and create a radial friction spring-force-type fit with the inside surface of the bore found on either the implant stem or broach. The flexion hole 68 may reduce the stress concentration and risers at the endpoint of the slit during flexion.

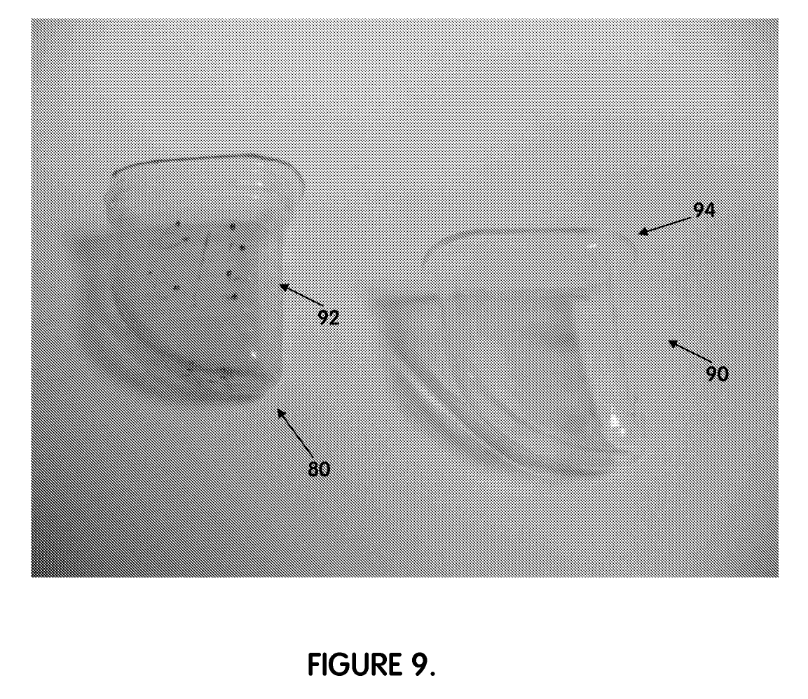

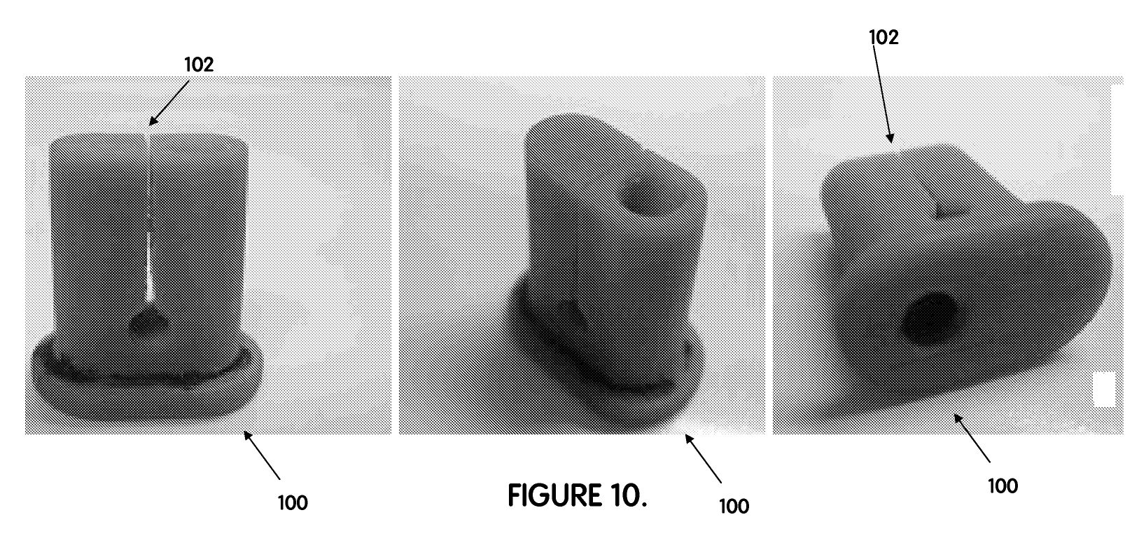

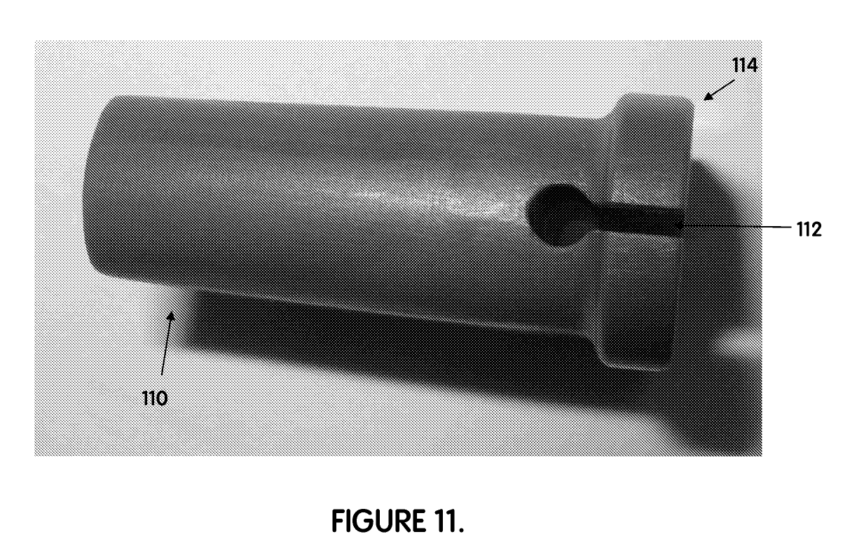

FIGS. 9 through 11 show alternate embodiments of a stem adapter 80, 90, 100, and 110. The embodiments may use different material (as shown in FIG. 9) that may conform with ridges 92 on the surface for grip as opposed to the flexion slit and hole. A flange 94 may conform to the flange depth of a stem inserter tool. Such an adaptor 80, 90 may be hollow, may be chamfered or radiused, and may be used in conjunction with other adaptors as protection of the bore.

The adaptor 100 may alternatively not have a radius or chamfer. It may be generally cylindrical around except for a flexion slit 102. The adaptor 100 may be inserted into the bore of a stem and taper within the bore to exert an outward force. Additionally, a slot 112 in a flange 114 of an adaptor 110 may snap in place when inserted into a stem inserter tool. Such an embodiment may allow for tolerances between the adaptor and the inserter tool.

While it is preferred that the stem adaptor of the present invention has a chamfer or radius on one of its prongs (e.g., as shown, the lateral prong) to promote easy removal via “levering” the tool, the stem adaptor does not need a radius to function. The stem adaptor may or may not utilize a flexion slit and/or flexion hole. While it may require more “wiggling” of the tool for insertion and extraction, a stem adaptor having a non-chamfered, non-radius lateral prong creates more operable surface engagement between the stem adapter and the stem's modular neck bore, thereby creating a slightly improved friction fit.

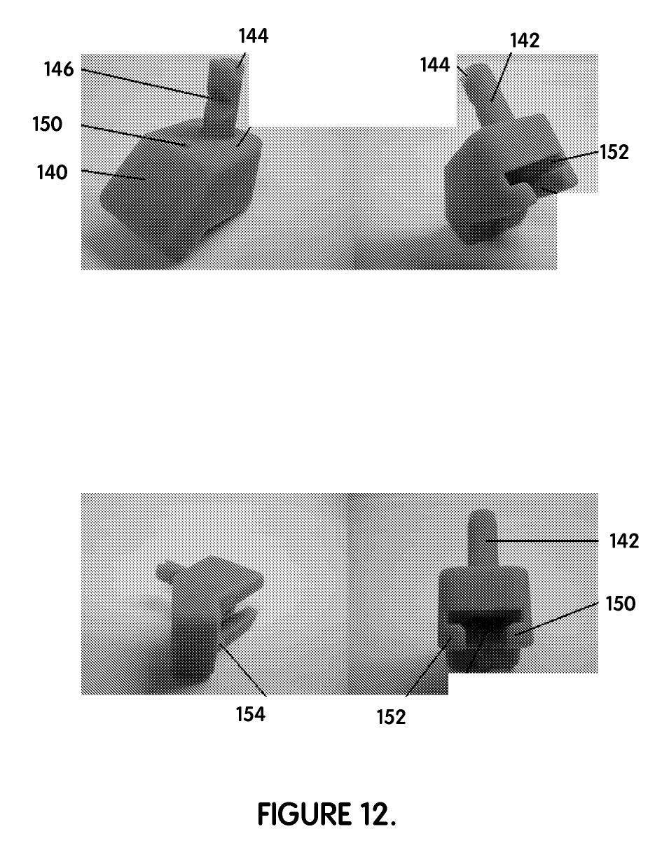

FIG. 12 shows views of a stem inserter 140. The stem inserter 140 generally serves to join a stem adaptor to a conventional broach handle. The stem inserter 140 generally eliminates the need for “new” (i.e., “extra”) insertion tools designed specifically for MIS/modular neck hip stems, since it utilizes a locking shank found on most primary system broaches.

The stem inserter 140 includes a post 142 having a locking groove 144 and a locking notch 146 attached to a body 150. A T-shaped groove 152 in the body receives a stem adaptor along a track extending into the body 150 of the inserter 140.

A stem adaptor is placed in the oval-tapered bore of an implant stem or postless broach, then mates with the T-shaped slot 152 of the stem inserter 150, and then connects to a conventional broach handle or other stem insertion tool. In other words, instrumentation may be connected in the manner: broach handle connected to the stem inserter (via the locking shank), which is connected to the stem adaptor (via the stem adaptor flange), which is connected to the stem of a broach, implant, or trial component through the taper lock.

The T-shaped slot 152 is configured to receive the flange of the stem adaptor. It is preferred that the stem inserter and/or the stem adaptor utilize a tactile “clicking” lock feature to inform a surgeon that the stem adaptor is rigidly locked in place within the stem inserter and will not come loose during use. An alignment numb 152 on the inserter 140 may align the inserter with the stem.

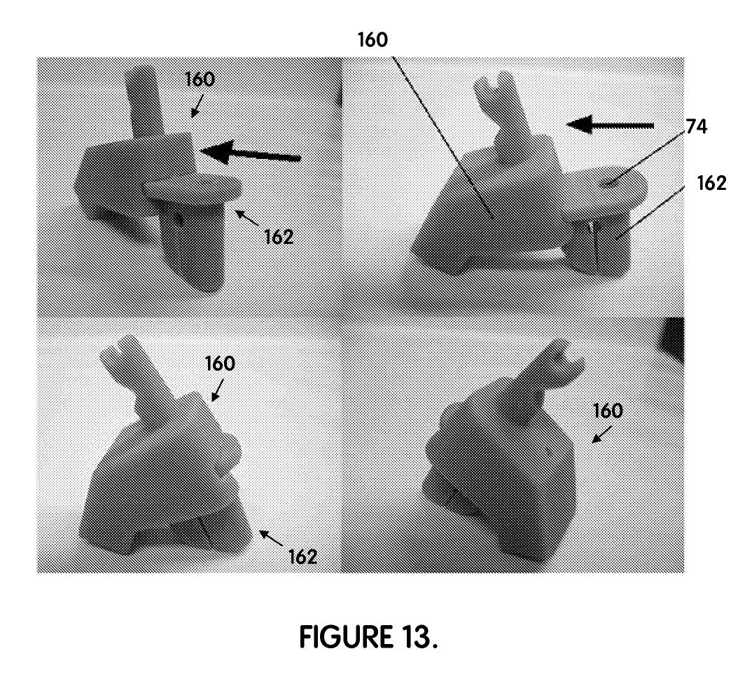

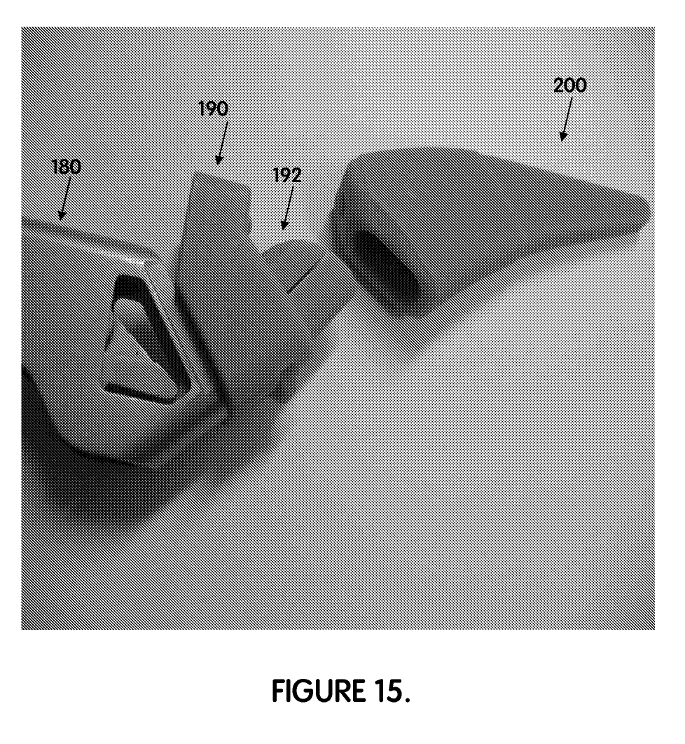

FIG. 13 shows views of a stem inserter 160 receiving a stem adaptor 162. The combination stem adaptor 162 and stem inserter 160 are used together in combination to connect a conventional inserter/extractor handle (typically used only with broach handles 170 having a conventional post/locking shank 172 (See FIG. 15)) to a broach which does not have a post or locking shank, but rather has a large recess configured to receive a modular-style neck. Without the combination provided, it would not be possible to take advantage of existing instrumentation with postless broaches.

The stem inserter/stem adaptor combination connects a conventional broach handle to the stem of a postless broach, implant, or trial and may be made disposable. While it is preferred that the postless broach inserter of the present invention utilizes a simple pivoting lever to catch an engagement recess to prevent removal, any mechanism known in the art such as cams, linkages, sliding track mechanisms, etc. . . . may be equally employed to serve the same function. The means for biasing the lever may comprise any one of but not limited to: torsion springs, compression springs, elastics, elastomeric plugs, and/or magnets.

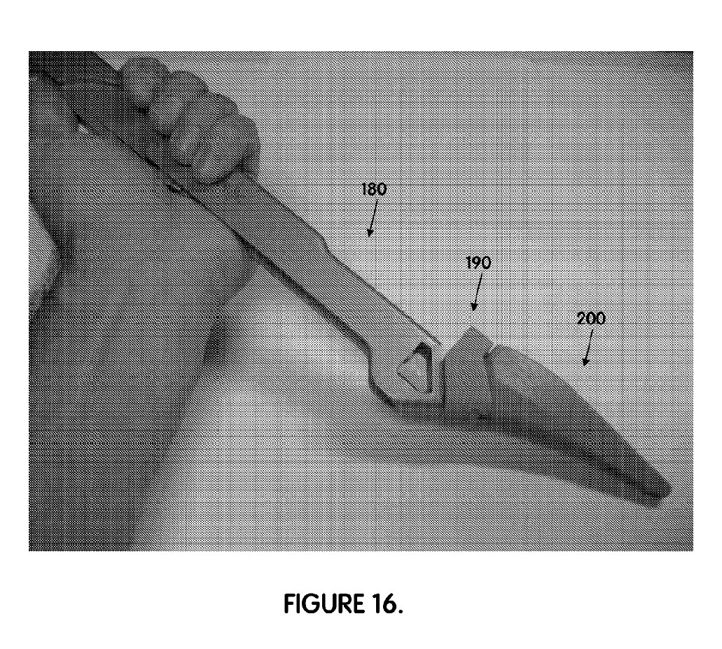

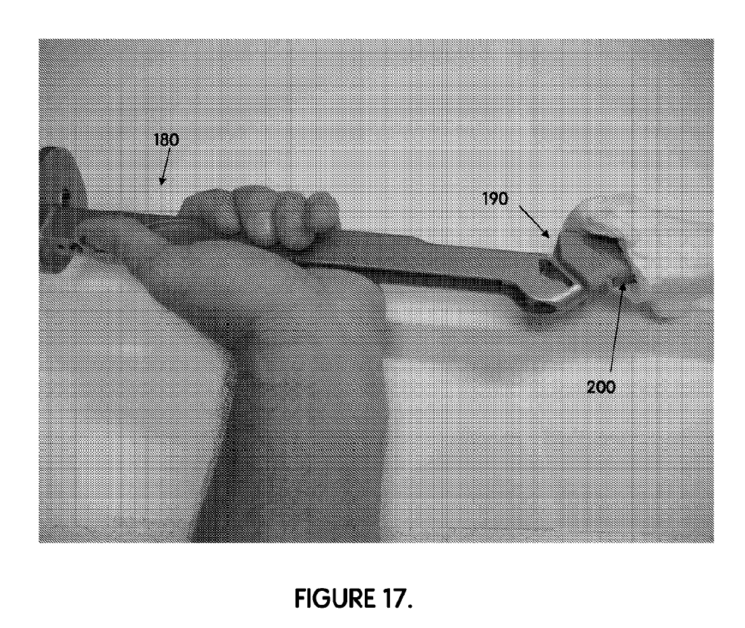

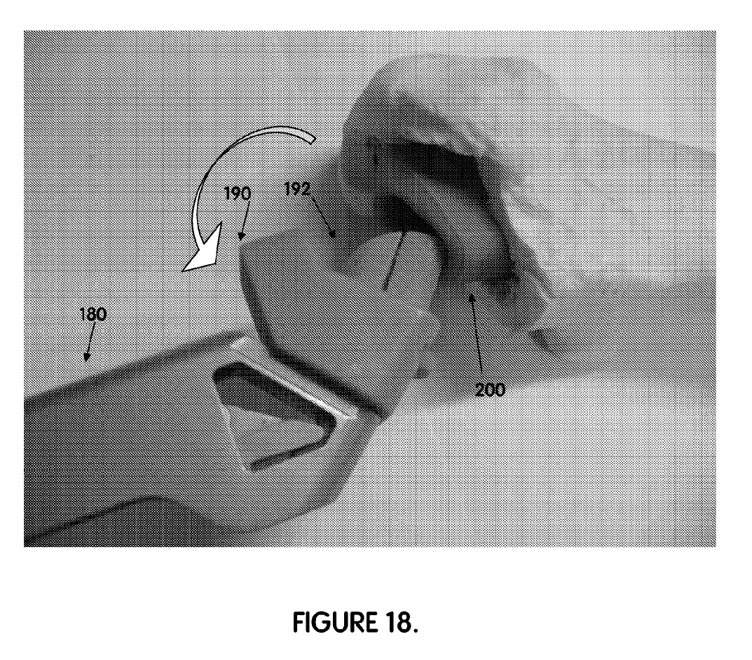

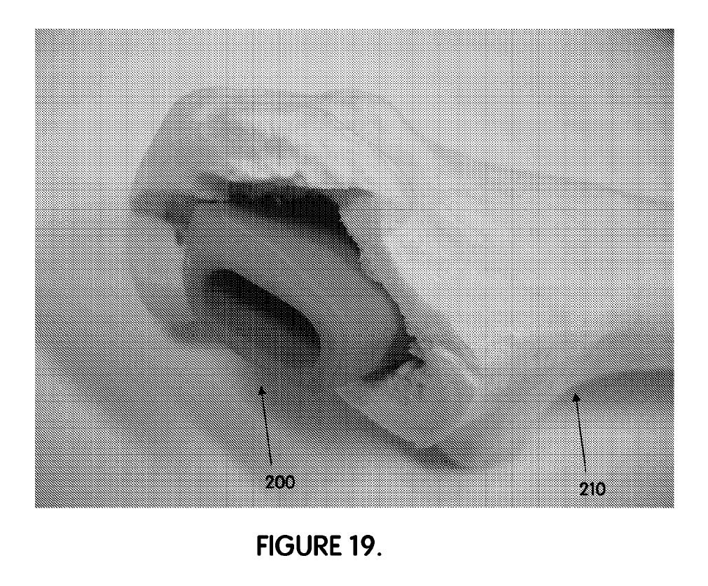

As shown in FIGS. 15 to 19, the stem inserter tool 180 may insert a stem 200 in a bone 210. FIG. 15 is a view of a stem inserter 190 and adaptor 192 engaged to a stem inserter tool 180 ready to be inserted into a stem 200. FIG. 16 is a view of a stem 200 attached to a stem inserter tool 180. FIG. 17 is a view of a stem 200 attached to a stem inserter tool 180, inserted in an intramedullary canal of the bone 210. FIG. 18 is a view of a stem adaptor 192 being removed from a stem 200. FIG. 19 is a view of a stem 200 properly inserted in an intramedullary canal of the bone 210.

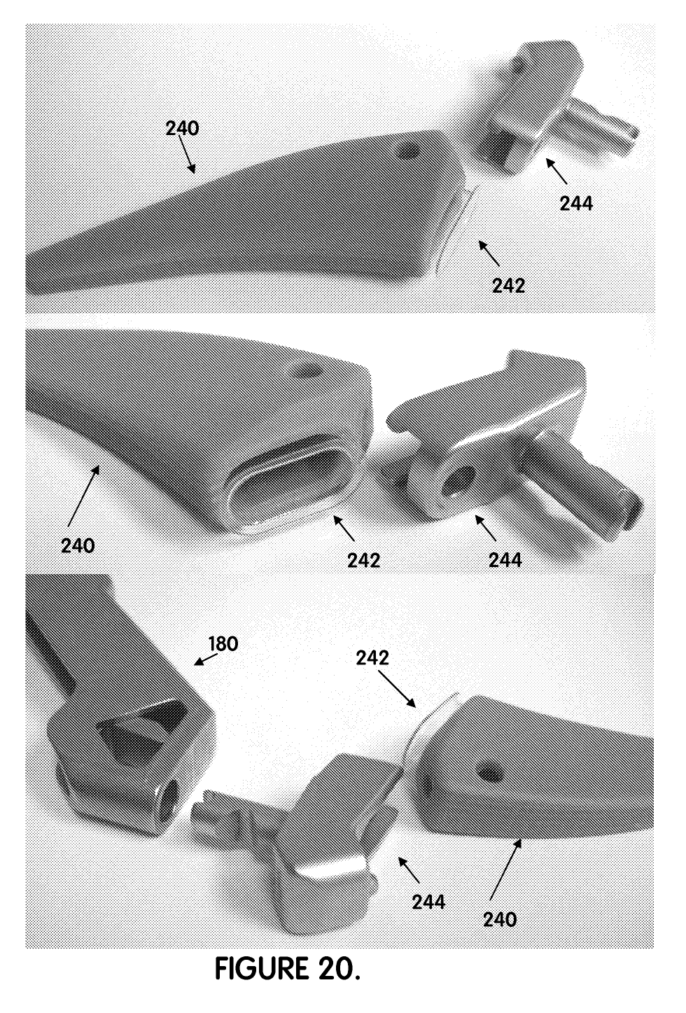

Turning now to FIG. 20, FIG. 20 shows views of a stem 240, an alternative stem adaptor 242, and a stem inserter 244. This combination, in use, is similar to the combinations of FIGS. 15 to 19. In all of these embodiments, the stem adaptor inserts into the stem. The stem inserter slidingly engages the stem adaptor. The nub on the stem adaptor can properly orient the inserter to the stem. A stem inserter tool is locked to the post of the stem inserter and then the stem may be placed in the bone. The stem adaptor may then be levered out of the stem across the chamfered surface by rotating the stem inserter tool relative to the stem. The stem would then by oriented in the bone and the instrumentation would be free from the stem.

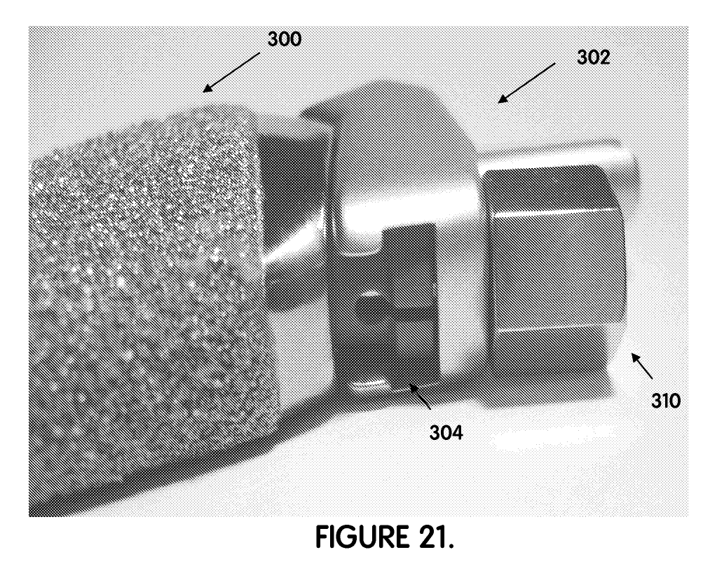

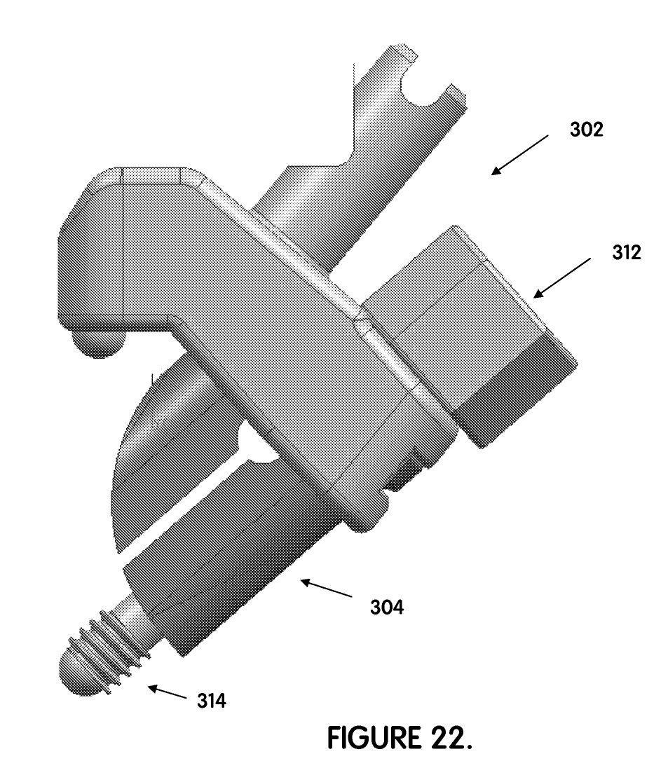

Turning now to the embodiments of FIGS. 21 and 22, FIG. 21 is a view of an alternate embodiment of a stem 300, stem inserter 302 and stem adaptor 304 including a fixation element 310. FIG. 22 is a view of the stem inserter 302 and stem adaptor 304 of FIG. 21. Together, the combination makes a rigid stem inserter. Rigid stem insertion comprises the above steps with respect to other stem inserters, but additionally includes fixing means 310 (e.g., a screw member, snap fastener, quarter-turn fastener, pull rivet, wedge, etc.) for enabling extraction of the stem and more positive interlocking therebetween.

Preferably, the fixing means comprises a screw member having a head 312 and threaded distal end 314 which may be placed through the bore of the medial prong of the stem adaptor when using the neck extractor, or may be placed through both the stem inserter and stem adaptor when using a conventional broach handle. The screw member 310 (or other fixing means) is then threaded into the stem or broach. The screw member may have a driving recess in its head which could be any one of a slot, Phillips socket, Allen hexagonal, Torx or other polygonal recess for use with a driving tool. Alternatively, the outer head surface may also have an external polygonal periphery for engagement by a socket or wrench-type tool. This outer engagement profile can be used as a backup means if the driving recess strips out.

All impact forces are seen on the proximal face of the stem or broach and the distal portion of the neck extractor. The stem adaptor and screw merely provide a connecting means rather than a force transmission means. The screw may be assembled to the stem adaptor and implant stem (or broach) prior to engaging the neck extractor tool, or may be inserted after the neck extractor tool, stem adaptor and implant stem/broach have been assembled.

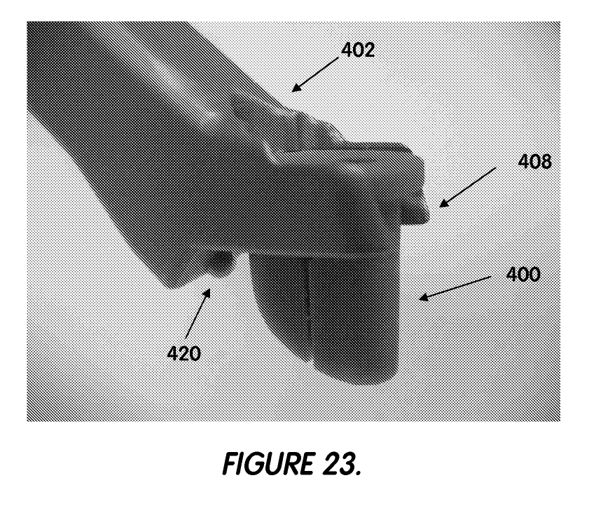

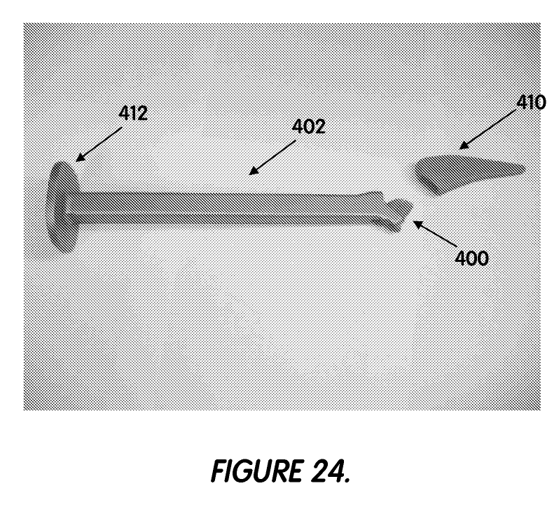

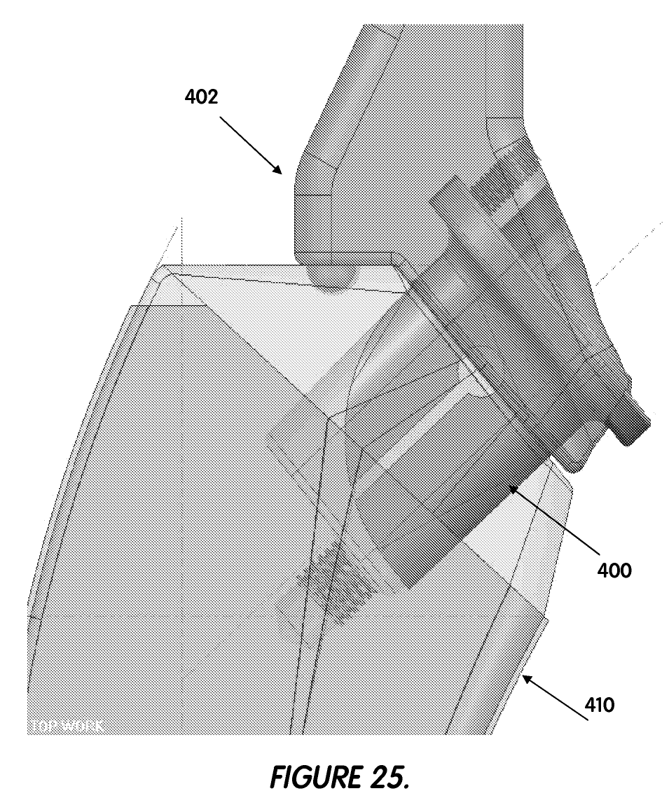

Turning now to FIGS. 23 to 25, FIG. 23 is a view of a stem adaptor 400 coupled to a stem extractor 402. FIG. 24 is a view of the stem adaptor 400 and neck extractor 402 of FIG. 23 and a stem 404. FIG. 25 is a see-through view of the stem adaptor 400 and neck extractor 402 of FIG. 23 coupled to a stem 406.

The stem adaptor is placed within a bi-forked distal end 410 of the neck extractor tool 402, so that a bore of the stem adaptor 400 is facing outwardly and is accessible in the event optional fixation means is required. The stem adaptor 400 slides between the fingers of the bi-forked end 408 until a tactile operable engagement is achieved. Tactile “snapping” engagement may be enabled by a slot in a flange of the stem adaptor 400, in combination with one or more lips on the inside of the bi-forked end 408 of the neck extractor 402. Using the stem adaptor 400, a broach or implant stem 410 may be mated to the neck extractor tool 402. The implant stem 410 or broach is now ready to be inserted into the femoral canal by tapping on a blunt end 412 of the neck extractor 402.

Preferably, the stem adaptor should be inserted with the radiused/chamfered prong first. The bore should be located towards the distal ends of the fingers. If the stem adaptor is inverted on accident, it will still work, and will hold the implant stem or broach to the neck extractor tool. However, removal in this misconfiguration may cause the neck extractor tool to contact the greater trochanter when “rolling” the neck extractor tool laterally instead of medially. This misconfiguration will also not enable a fixing means such as a screw member for rigid stem insertion as will be described later and is illustrated in the drawings.

The preferred positioning for the stem adaptor within the neck extractor fingers is illustrated in the figures. An alignment nub 420 is facing the chamfered/radiused prong of the stem adaptor. Also notice that the proximal bore end is visible, is located toward the finger ends, and is easily accessible for screw insertion.

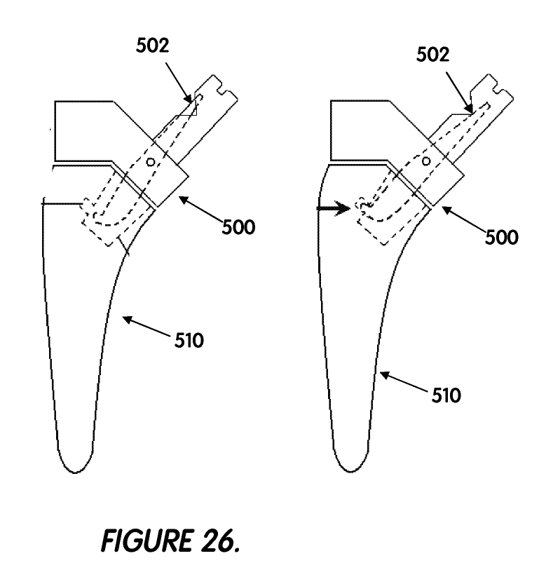

Turing now to FIG. 26, FIG. 26 shows views of an alternate embodiment of a stem inserter 500 with a positive mechanical engagement element 502. The postless inserter 500 closely resembles other stem inserters, however, it has provided thereon, a biased mechanism 502 that operates upon engagement with a conventional broach handle. The postless broach inserter 500 first is placed into the bore of a postless broach (i.e., a broach having a bore adapted to receive a modular neck). A conventional broach handle then secures itself to a locking shank 504 of the postless broach inserter 500 in the conventional manner. During engagement of the broach handle to the postless broach inserter 500, a mechanism 502 is activated, the mechanism 502 serving as means for locking the conventional broach handle to a postless broach 510.

The postless broach inserter also allows trialing with modular-style necks to take place with the postless broach in-situ. The advantage of trialing with the broach still in the femoral canal, is that trial stems are not necessary, and therefore excessive bone removal from repetitive scratch fitting within the medullary canal is virtually eliminated. In other words, the broach does not have to be removed to insert one or more trial stems, which will then also need to be removed prior to implanting the final prosthesis stem. Additionally, postless broaches provided by the present invention may utilize the same trial necks as the implants, thereby removing any need for a separate set of trial necks that fit the instrumentation. By cutting down on the number of parts in the instrumentation system, overhead, labor, and operating room time is reduced.

When a conventional broach handle locks into the locking notch of the locking shank of the postless broach inserter, a lever (or other equivalent mechanism such as an axially-displaceable rivet mandrel) is displaced from its biased position and pivots such that a distal end of said lever engages a side recess within a bore of the postless broach. This engagement stabilizes the postless broach inserter onto the broach in at least an approximate axial direction and approximate radial direction, making both insertion and removal of the postless broach possible, even though the postless broach does not have an external locking shank. The postless broach inserter may alternatively be used with implant stems or trial implants, that is, if the implant stems are also configured with a side recess within its oval-tapered bore. The portion of the lever that enters the bore of the implant stem or postless broach may be shaped so as to compliment the bore and provide an alignment feature.

Once the broach is properly inserted within the femoral canal, the broach handle can be removed, and subsequently, the postless broach inserter may be removed. Trialing using modular necks and various head sizes can ensue thereafter. Once trialing is complete, the postless broach inserter is placed back into the bore of the broach, and the broach handle is engaged with the postless broach inserter to lock the broach, broach handle, and postless broach inserter together as one integral piece once again. The broach handle is then tapped in reverse to remove the entire assembly from the femoral canal. It should be noted that the side recess may be located anywhere within the bore and have multiple configurations without departing from the scope.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, any one instrument of the present invention may share features of another without limitation. For example, the postless broach inserter may also comprise a screw hole extraction means similar to the stem adaptor and stem inserter combination for additional holding strength.

Thus, the breadth and scope of the present invention should not be limited by any of the above-described exemplary embodiments, but should be defined only in accordance with the following claims appended hereto and their equivalents.

What is claimed is:

1. Instrumentation for a modular prosthesis having a stem and a neck, the neck being configured to be received within a bore of the stem, comprising: a. a handle extending along an axis, the handle having a distal end and a proximal end, the proximal end having a blunt surface configured to be struck; and b. an adaptor having a male end configured to mate inside the bore of the stem, and further having a surface configured to mate with the distal end of the handle; c. wherein the adaptor is configured to have a medial portion and a lateral portion, the medial portion having a relief such that the adaptor is configured to disengage the stem when the adaptor is rotated relative the stem.

2. The instrumentation of claim 1, wherein the relief is a radius.

3. The instrumentation in claim 1, wherein the distal end extends from the handle at an angle to the long axis.

4. The instrument of claim 3, wherein the distal end of the handle is configured to have a slot extending away from the long axis and the adaptor surface configured to mate with the distal end of the handle is a flange.

5. The instrument of claim 4, wherein the adaptor is further configured to have a slit between the medial portion and the lateral portion, the slit configured to flex the medial and lateral portions of the adaptor such that the adaptor is wedged within the bore when the adaptor is inserted within the bore.

6. The instrument of claim 1, wherein the handle is an extraction tool.

7. The instrument of claim 1 wherein the handle is an insertion tool.

8. The instrument of claim 1 wherein the adaptor is a hollow body.

9. An extraction tool for extracting a modular neck from a stem of a prosthetic device, comprising: a. A proximal end having a blunt surface configured to be struck; b. a shank extending along an axis away from the proximal end; c. a distal end extending from the shank at an angle to the long axis, the distal end being forked such that a pair of prongs on the fork may engage the modular neck seated in the stem, the prongs having a first distance at a point close to the long axis and a second distance greater than the first distance at a point away from the long axis relative to the first point.

10. The extraction tool of claim 9, wherein the distal end narrows as the distal end extends away from the shank.

11. The extraction tool of claim, wherein the extraction tool wedges the neck between the prongs when the blunt surface is struck.

12. The extraction tool of claim 11, wherein the extraction tool delivers a first force to the stem generally along the long axis of the handle and a second force between the extraction tool and the neck generally perpendicular to the long axis such that the neck is forced away from the stem.

13. The extraction tool of claim 11 wherein the extraction tool is rotated relative to the stem to force the neck away from the stem.

14. A method for performing a prosthetic surgery including a modular stem having a bore configured to receive a neck component, comprising the steps of:
a. coupling the bore of the modular stem to a male mating surface on a distal end of a first elongated tool having a blunt proximal end configured to be struck;
b. inserting the stem into a reamed bone canal;
c. striking the blunt proximal end of the tool to seat the stem; and
d. rotating the first elongated tool relative to the stem to decouple the tool from the stem.

15. The method of claim 14, further comprising the step of: a. removing a neck component from the stem by striking the blunt proximal end of the tool thereby delivering a first force to the stem generally along a long axis of the tool and a second force between the tool and the neck component generally perpendicular to the long axis such that the neck is forced away from the stem.

16. The method of claim 15, further comprising the step of wedging the tool against the neck component by striking the blunt proximal end.

17. The method of claim 14, further comprising the step of removing a neck component from the stem by rotating the tool relative to the stem.
